(19) United States
(12) Patent Application Publication
(10) Pub. No.: US 2026/0043036 A1
(43) Pub. Date: Feb. 12, 2026

(54) NEEDLE-LADEN PLANT INOCULATION METHOD

(71) Applicant: The Regents of the University of California, Oakland, CA (US)

(72) Inventors: Nicole Steinmetz, San Diego, CA (US); Andrea Monroy-Borrego, La Jolla, CA (US)

(21) Appl. No.: 19/099,018

(22) PCT Filed: Jul. 27, 2023
(86) PCT No.: PCT/US2023/028883

§ 371 Date: Jan. 27, 2025

Related U.S. Application Data
(60) Provisional application No. 63/393,602, filed on Jul. 29, 2022.

(51) Int. Cl. C12N 15/82

(52) U.S. Cl. CPC C12N 15/8203; C12N 2770/00043

(57) ABSTRACT

Provided are improved methods for gene delivery to plants comprising injecting a polynucleotide, nucleoprotein complex, nanoparticle or plant viral nanoparticle into a stem or a petiole of the plant, thereby in it into the vascular system of the plant.

### CROSS-REFERENCE TO RELATED PATENT APPLICATION

This application claims the benefit under 35 U.S.C. § 119 (e) of U.S. Provisional Application No. 63/393,602, filed Jul. 29, 2022, the entire contents of which are incorporated herein by reference.

### STATEMENT OF GOVERNMENT SUPPORT

This invention was made with government support under AI161306 awarded by the National Institutes of Health (NIH), DMR-2011924 awarded by the National Science Foundation (NSF), and 2020-67021-31255 awarded by the United States Department of Agriculture (USDA). The government has certain rights in the invention.

### BACKGROUND

Agriculture is facing new challenges, with global warming modifying the survival chances for crops, and new pests on the horizon. To keep up with these challenges, gene delivery provides tools to increase crop yields. On the other hand, gene delivery also opens the door for molecular farming of pharmaceuticals in plants. However, towards increased food production and scalable molecular farming, there remain technical difficulties and regulatory hurdles to overcome. The industry-standard is transformation of plants via Agrobacterium tumefaciens, but this method is limited to certain plants, requires set up of plant growth facilities and fermentation of bacteria, and introduces lipopolysaccharides contaminants into the system. Therefore, alternate methods are needed.

To increase food production and advance molecular farming, there is a need for improved gene delivery methods for plants. Traditional stable genetic transformation is mostly done using plant callus and Agrobacterium tumefaciens-mediated or biolistic particle delivery, with the relatively new short palindromic repeats (CRISPR) associated protein 9 (Cas9) technique being explored. However, transgenic plant engineering is costly and it requires long time periods to develop a desirable organism (i.e., the timeframe from callus to a developed plant can take months). Other challenges are that transformation yields may be low or result in undesirable variations; in addition, tight regulations of genetically modified organisms (GMOs) limit their scope for application.

Accordingly, a need exists in the art for improved gene delivery methods. This disclosure satisfies this need and provides related advantages as well.

### SUMMARY OF THE DISCLOSURE

An aspect of the disclosure is directed to a method for inserting a polynucleotide, a nucleoprotein complex, a nanoparticle or a plant viral nanoparticle into the vascular system of a plant, comprising, or alternatively consisting essentially of, or yet further consisting of injecting the polynucleotide, nucleoprotein complex, nanoparticle or plant viral nanoparticle into a stem or a petiole of the plant, thereby inserting it into the vascular system of the cell.

### BRIEF DESCRIPTION OF THE FIGURES

FIGS. 1A-1B: show schematic representation of the experimental design. TMV was used as a model to test mechanical, spray, and syringe inoculation (FIG. 1A) Photographs demonstrating the applications of TMV via mechanical, spray, and syringe inoculation (FIG. 1B) Some icons shown are from BioRender.com.

FIGS. 2A-2D: show photographic documentation of the injection of fluorophore Oregon Green 488™ into the petiole. (FIG. 2A, FIG. 2B) Plants imaged under UV light. (FIG. 2C, FIG. 2D) Photograph of TMV injection into the petiole.

FIGS. 3A-3E: show photographs after injection of the fluorophore Oregon Green 488™ in the plant's stem. (FIG. 3A, FIG. 3B) Plants imaged under UV light. (FIG. 3C) Photographic documentation of TMV injection into the stem. (FIG. 3D) After the inoculation, early signs of necrotic tissue were observed in the stem, on average 8 days post infection, (FIG. 3E) which would expand causing leaf necrosis and death. Therefore, viral syringe inoculation into the stem is not a suitable method for an infectious vector—it may be suitable for non-infectious nanoparticles.

FIGS. 4A-4E: show representative photographs of N. benthamiana from day 0 to day 10-15, (FIG. 4A) control non-infected plants, and TMV days post inoculation (dpi) by (FIG. 4B) mechanical, (FIG. 4C) spray, and (FIG. 4D) syringe method using 3, 15, and 30 μg of TMV. (FIG. 4E) Graphed yields, and a table of the values for the TMV yields obtained. The experiments were done in duplicate using 10 plants per treatment.

FIG. 5: shows representative symptoms of N. benthamiana infected with TMV-Lys. Phenotype 1, shows generalized yellow mottling of the leaves increasing with time. Phenotype 2, shows the blackening of veins with the simultaneous yellowing of the leaves.

FIG. 6: shows graph and table showing the yields of TMV-Lys by the different inoculation methods (mechanical, spray, syringe) using 3 or 30 μg of the plant virus. For groups Y1, Y2, and Y3 the data was obtained by extracting TMV-Lys from a single plant (open symbols)—the yields were normalized to 100 grams of leaves for comparison with pooled samples (Y4, filled symbols). Group Y4 are yields from pooled leaves from 10 plants. Data is in good agreement regardless of the sample size.

FIGS. 7A-7B: show TEM images of TMV and TMV-Lys obtained by different inoculation methods, scale bar represents 200 nm (FIG. 7A). SDS-PAGE of the extracted TMV and TMV-Lys, the presence of its coat protein (17.5 kDa) was consistent in all samples. (FIG. 7B) SDS-PAGE analysis was consistent with the presence of pure TMV preparations showing the 17.5 kDa TMV coat protein; plant contaminants were not apparent; also, contamination of control plants was also not detected.

FIG. 8: shows photographic documentation of the effects of Silwet L-77 spray on N. benthamiana 24 hours post treatment. 4-5 weeks old plants tolerated concentrations below 0.04% Silwet L-77. Silwet L-77 treatment caused discoloration and darkening of the leaves which was prevalent at 0.4% Silwet L-77. Using 4% Silwet L-77 caused necrotic tissue. Older plants (6-7 weeks old) were more resistant to the effects of the surfactant, with almost no adverse effects observed for up to a 0.4% of Silwet L-77 (higher concentrations were not tested).

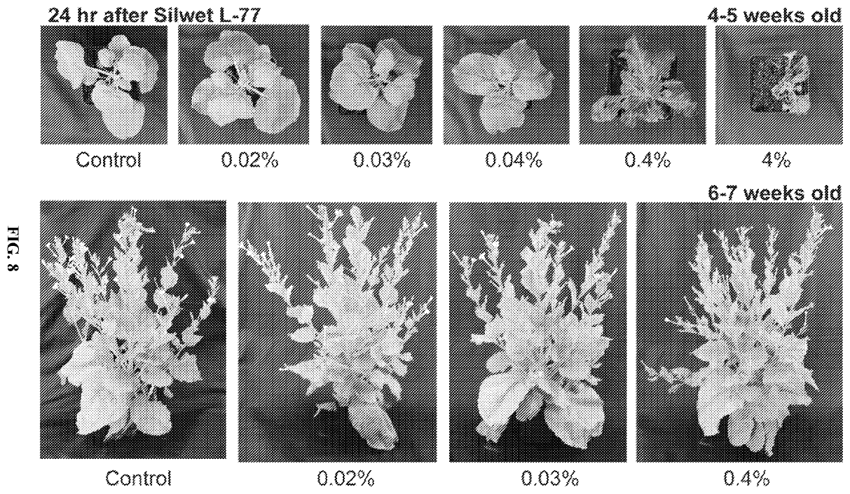

FIG. 9: shows photographs of N. benthamiana from day 0 to day 15 TMV-Lys days post inoculation (dpi) via mechanical [3, 30 μg], spray [3, 30 μg], and syringe [3, 30 μg] inoculation using TMV-Lys. The experiments were done in duplicate and 10 plants were used per treatment.

FIG. 10: shows representative photographs of N. benthamiana from day 0 to day 15 post inoculation—these are the negative controls: mechanical control are plants rubbed with carborundum, spray control is treated with 0.03% Silwet L-77, and the syringe control was injected with NaPB at the petiole and stem.

### DETAILED DESCRIPTION OF THE DISCLOSURE

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications may be referenced by an identifying citation or by an Arabic numeral or first author name. The full citation for the publications identified by an Arabic numeral or first author name are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988)) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

As used herein, “biologics” or “biological drug” and their plurals are used interchangeably and refer to drugs consisting of or comprising biological molecules or material, i.e., proteins, polypeptides, peptides, polynucleotides, oligonucleotides, polysaccharides, oligosaccharides and fragments thereof, as well as cells, tissues, biological fluids or extracts thereof. In some embodiments, biological drugs may include proteins such as monoclonal antibodies, cytokines, soluble receptors, growth factors, hormones, enzymes, adhesion molecules and fusion proteins and peptides that are specific to certain targets known to modulate disease mechanisms. In yet some further embodiments, biological drugs may include or target any component participating in molecular and/or cellular processes such as, cell cycle, cell survival, apoptosis, immunity and the like. In more specific embodiments, biological drugs may be any checkpoint protein/s or any modulators or inhibitors thereof, or any combinations thereof. In yet some further embodiments, biological drugs (or their precursors or components) may be isolated from living sources human, animal, plant, fungal, or microbial.

Still further in some embodiments, “biologics” refers to a class of therapeutics that are produced by means of biological processes involving recombinant DNA technology which are usually one of three types: (a) substances that are similar to the natural occurring proteins: (b) monoclonal antibodies; and (c) receptor constructs or fusion proteins, usually based on a naturally occurring receptor linked to the immunoglobulin frame.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. (1987)) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. (1987)) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

A “marker gene” or a “selectable marker gene” refers to a gene whose expression in a plant cell gives the cell a selective advantage. The selective advantage possessed by the cells transformed with the marker gene may be due to their ability to grow in presence of a negative selective agent, such as an antibiotic or a herbicide, compared to the ability to grow of non-transformed cells. The selective advantage possessed by the transformed cells may also be due to their enhanced capacity, relative to non-transformed cells, to utilize an added compound as a nutrient, growth factor or energy source. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

As used herein, a “nucleoprotein complex” is a complex comprising proteins conjugated with nucleic acids (either DNA or RNA).

As used herein, the term “nanoparticle” refers to particles that are on the order of 10⁻⁹ or one billionth of a meter and below 10-6 or 1 millionth of a meter in size. The term “nanoparticle” includes nanospheres; nanorods; nanoshells; and nanoprisms; and these nanoparticles may be part of a nanonetwork. The term “nanoparticles” also encompasses liposomes and lipid particles having the size of a nanoparticle. The particles may be, e.g., monodisperse or polydisperse and the variation in diameter of the particles of a given dispersion may vary, e.g., particle diameters of between about 0.1 to 100's of nm.

In some embodiments, the nanoparticle is of size about 1 nm to about 1000 nm, about 50 nm to about 500 nm, about 100 nm to about 250 nm, or about 200 nm to about 350 nm. In one embodiment, the nanoparticle is of about 100 nm to about 1000 nm. In another embodiment, the nanoparticle is of size about 80 nm to about 200 nm. In one embodiment, nanoparticle is of size about 50 nm to about 500 nm. In some embodiments, nanoparticle is of size about 158 nm, about 218 nm, or about 305 nm. In some embodiments, nanoparticle is of size about 337 nm, about 526 nm, about 569 nm, about 362 nm, about 476 nm, about 480 nm, about 676 nm, about 445 nm, about 434 nm, about 462 nm, about 492 nm, about 788 nm, about 463 nm, or about 65 nm.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.”

The “petiole” is the stalk of a plant that supports a leaf and attaches it to the stem. The “stem” is the part of the plant that serves as the main source of support and produces nodules and roots, which is not observed in petioles.

As used herein, the term “plasmid/protein complex” intends a plasmid encoding a biologic mixed with a plant virus coat protein or capsid.

### Viral Plant Nanoparticles (VPNs)

Viral plant nanoparticles (VPNs, or plant viral nanoparticles) are generally composed of one or more viral proteins, such as, but not limited to, those proteins referred to as capsid, coat, shell, surface and/or envelope proteins, or particle-forming polypeptides derived from these proteins. VPNs can form spontaneously upon recombinant expression of the protein in an appropriate expression system. VPNs can also be engineered, e.g., comprising, or consisting essentially of, or yet further consisting of, one or more viral proteins that comprise, or consists essentially of, or yet further consists of, a modification. Methods for producing VPNs are known in the art. The presence of VPNs following recombinant expression of viral proteins can be detected using conventional techniques known in the art, such as by electron microscopy, biophysical characterization, and the like. Further, VPNs can be isolated by known techniques, e.g., density gradient centrifugation and identified by characteristic density banding. See, for example, Baker et al. (1991)) Biophys. J. 60:1445-1456; and Hagensee et al. (1994)) J. Viral. 68:4503-4505; Vincente, J Invertebr Pathol., (2011)); Schneider Ohrum and Ross, Curr. Top. Microbial. Immunol., 354:53073, (2012)).

In some embodiments, the virus or VPN is derived from Cowpea chlorotic mottle virus (CCMV). CCMV is a spherical plant virus that belongs to the Bromovirus genus. Several strains have been identified and include, but not limited to, Carl (Ali, et al., (2007). J. Virological Methods 141:84-86), Car2 (Ali, et al., (2007). J. Virological Methods 141:84-86, (2007), type T (Kuhn, (1964). Phytopathology 54:1441-1442), soybean(S) (Kuhn, (1968). Phytopathology 58:1441-1442), mild (M) (Kuhn, (1979). Phytopathology 69:621-624), Arkansas (A) (Fulton, et al., (1975). Phytopathology 65:741-742), bean yellow stipple (BYS) (Fulton, et al., (1975). Phytopathology 65:741-742), R (Sinclair, ed. (1982). Compendium of Soybean Diseases. 2^{nd} ed. The American Phytopathological Society, St. Paul. 104 pp.), and PSM (Paguio, et al., (1988). Plant Diseases 72 (9): 768-770).

In some instances, the virus or VPN from CCMV comprise, or consists essentially of, or yet further consists of, a plurality of capsid proteins. In some instances, the capsid protein is a wild-type CCMV capsid, optionally expressed by Car1, Car2, type T, soybean(S), mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprise, or consists essentially of, or yet further consists of, the sequence as set forth in the UniProtKB ID P03601:

(SEQ ID NO: 1)
MSTVGTGKLTRAQRRAAARKNKRNTRVVQPVIVEPIASGQGKAIKAWTG
YSVSKWTASCAAAEAKVTSAITISLPNELSSERNKQLKVGRVLLWLGLL
PSVSGTVKSCVTETQTTAAASFQVALAVADNSKDVVAAMYPEAFKGITL
EQLTADLTIYLYSSAALTEGDVIVHLEVEHVRPTFDDSFTPVY,
or
an equivalent thereof.

In some cases, the virus or VPN from CCMV is prepared by the method as described in Ali et al., “Rapid and efficient purification of Cowpea chlorotic mottle virus by sucrose cushion ultracentrifugation,” Journal of Virological Methods 141:84-86 (2007).

In some embodiments, the virus or VPN is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the virus or VPN from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: 2)
MEQNLFALSLDDTSSVRGSLLDTKFAQTRVLLSKAMAGGDVLLDEYLYD
VVNGQDFRATVAFLRTHVITGKIKVTATTNISDNSGCCLMLAINSGVRG
KYSTDVYTICSQDSMTWNPGCKKNFSFTFNPNPCGDSWSAEMISRSRVR
MTVICVSGWTLSPTTDVIAKLDWSIVNEKCEPTIYHLADCQNWLPLNRW
MGKLTFPQGVTSEVRRMPLSIGGGAGATQAFLANMPNSWISMWRYFRGE
LHFEVTKMSSPYIKATVTFLIAFGNLSDAFGFYESFPHRIVQFAEVEEK
CTLVFSQQEFVTAWSTQVNPRTTLEADGCPYLYAIIHDSTTGTISGDFN
LGVKLVGIKDFCGIGSNPGIDGSRLLGAIAQ,
or
an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprise, or consists essentially of, or yet further consists of, the sequence as set forth in the UniProtKB ID P03599 (residues 834-1022):

(SEQ ID NO: 3)
GPVCAEASDVYSPCMIASTPPAPFSDVTAVTFDLINGKITPVGDDNWNT
HIYNPPIMNVLRTAAWKSGTIHVQLNVRGAGVKRADWDGQVFVYLRQSM
NPESYDARTFVISQPGSAMLNFSFDIIGPNSGFEFAESPWANQTTWYLE
CVATNPRQIQQFEVNMRFDPNFRVAGNILMPPFPLSTETPPL,
or
an equivalent thereof.

In some embodiments, the virus or VPN is derived from Physalis mottle virus (PhMV). PhMV is a single stranded RNA virus that belongs to the genus Tymovirus. In some instances, the virus or VPN from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprise, or consists essentially of, or yet further consists of, the sequence as set forth in the UniProtKB ID P36351:

(SEQ ID NO: 4)
MDSSEVVKVKQASIPAPGSILSQPNTEQSPAIVLPFQFEATTFGTAETA
AQVSLQTADPITKLTAPYRHAQIVECKAILTPTDLAVSNPLTVYLAWVP
ANSPATPTQILRVYGGQSFVLGGAISAAKTIEVPLNLDSVNRMLKDSVT
YTDTPKLLAYSRAPTNPSKIPTASIQISGRIRLSKPMLIAN,
or
an equivalent thereof.

In some embodiments, the virus or VPN is derived from Sesbania mosaic virus (SeMV). SeMV is a positive stranded RNA virus that belongs to the genus Sobemovirus. In some instances, the virus or VPN from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprise, or consists essentially of, or yet further consists of, the sequence as set forth in the UniProtKB ID Q9EB06:

(SEQ ID NO: 5)
MAKRLSKQQLAKAIANTLETPPQPKAGRRRNRRRQRSAVQQLQPTQAGI
SMAPSAQGAMVRIRNPAVSSSRGGITVLTHSELSAEIGVTDSIVVSSEL
VMPYTVGTWLRGVAANWSKYSWLSVRYTYIPSCPSSTAGSIHMGFQYDM
ADTVPVSVNQLSNLRGYVSGQVWSGSAGLCFINGTRCSDTSTAISTTLD
VSKLGKKWYPYKTSADYATAVGVDVNIATPLVPARLVIALLDGSSSTAV
AAGRIYCTYTIQMIEPTASALNN,
or
an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a virus or VPN.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: nebi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid. \Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a virus or VPN described herein further comprise, or consists essentially of, or yet further consists of, a label or a tag, e.g., such as a detectable label. A detectable label can be attached to, e.g., to the surface of a virus or VPN.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷² As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the virus or VPN or nanoparticle. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

As used herein, the phrase “vascular system of a plant” refers to the assemblage of conducting tissues and associated supportive fibres that transport nutrients and fluids throughout the plant body in vascular plants. The two primary vascular tissues are xylem, which transports water and dissolved minerals from the roots to the leaves, and phloem, which conducts food from the leaves to all parts of the plant.

As used herein, the term “injecting” refers to driving or forcing a liquid into a person, animal or plant's body using a needle, a syringe or a similar device (e.g., a pump).

As used herein, the term “petiole” refers to the stalk of a leaf which attaches the blade to the stem.

As used herein, the term “stem” refers to the plant axis that bears buds and shoots with leaves and, at its basal end, roots.

As used herein, the phrase “polymer-condensed plasmid” refers to a plasmid packed in the presence of a polymer. In some embodiments, the polymer is a cationic polymer. In some embodiments, the polymer is poly-L-lysine (PLL).

As used herein, a “transgene” (a “foreign” gene, or an “exogenous” gene, or a “heterologous” gene) refers to a gene that is introduced into a host cell or organism by gene transfer.

### Modes For Carrying Out the Disclosure

Provided herein is a method for inserting a polynucleotide, a nucleoprotein complex, a nanoparticle or a plant viral nanoparticle (VPN) into the vascular system of a plant, comprising, or consisting essentially of, or yet further consisting of, injecting the polynucleotide, nucleoprotein complex, the nanoparticle or plant viral nanoparticle into a stem or a petiole of the plant, thereby inserting it into the vascular system of the cell. The polynucleotide comprises DNA or RNA.

Non-limiting examples of the polynucleotide include a nucleoprotein complex, a nanoparticle or plant viral nanoparticle, optionally a plasmid/protein complex, plasmid, polymer-condensed plasmid, or viral vector. Non-limiting examples of viral vectors include those that are derived from tobacco mosaic virus (TMV), cowpea mosaic virus, or potato virus X. Non-limiting examples of plasmids include those that are, or are derived from an agrobacterium Ti (tumor inducing) plasmid, a plant viral-derived expression plasmid (based on tobacco mosaic virus (TMV), cowpea mosaic virus or potato virus X).

The polynucleotide can comprise further elements, such a transgene or a polynucleotide encoding a transgene, optionally a marker gene, a therapeutic gene, a biologics, or a nanoparticle.

Non-limiting examples of plants include N. benthamiana, N. tabacum, or black eyed peas, or Arabidopsis thaliana.

An aspect of the disclosure is directed to a method for inserting a polynucleotide, a nucleoprotein complex, a nanoparticle or a plant viral nanoparticle into the vascular system of a plant, comprising or consisting essentially of, or yet further consisting of injecting the polynucleotide, nucleoprotein complex, nanoparticle or plant viral nanoparticle into a stem or a petiole of the plant, thereby inserting it into the vascular system of the cell.

In one aspect, the method comprises, or consists essentially of, or yet further consists of contacting and inserting the polynucleotide, nucleoprotein complex, or plant viral nanoparticle with a needle syringe into the vascular of the plant, such as a needle syringe that comprises from about 8 mm×31 G, or any needle that penetrates/can penetrate the tissue can potentially be used.

In some embodiments, the polynucleotide is DNA or RNA. In some embodiments, the polynucleotide is a nucleoprotein complex, nanoparticle or plant viral nanoparticle, optionally a plasmid/protein complex, plasmid, polymer-condensed plasmid, or viral vector, with an optional linker, further optionally a Lys linker.

In some embodiments, the viral vector is or is derived from tobacco mosaic virus (TMV), cowpea mosaic virus, or potato virus X.

In some embodiments, the plasmid is or is derived from an agrobacterium Ti (tumor inducing) plasmid, a plant viral-derived expression plasmid (based on tobacco mosaic virus (TMV), cowpea mosaic virus or potato virus X).

In some embodiments, the plant is N. benthamiana, N. tabacum, or Vigna unguiculata (black eyed pea), or Arabidopsis thaliana.

In some embodiments, the injection of the polynucleotide comprises contacting and inserting the polynucleotide, nucleoprotein complex, or plant viral nanoparticle with a needle syringe into the vascular of the plant.

The needle or syringe can be loaded with from about 1 μg to about 50 μg (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg) of polynucleotide per mL of carrier. Non-limiting examples of carriers include a buffer solution at a pH of from about 7.2 to about 7.6, or alternatively about 7.4.

After injection, the method can further comprise growing the plant until expression is established, 3-10 (e.g., 3, 4, 5, 6, 7, 8, 9, or 10) days post inoculation with polynucleotide or nucleoprotein complex or 7-20 (e.g., 7, 8, 9, 10, 11, 12, 13, 14, 15, 16, 17, 18, 19, or 20) days when inoculating with a nanoparticle or plant viral nanoparticle, and then further isolating the polynucleotide, the nucleoprotein complex, the nanoparticle or the plant viral nanoparticle from the plant cell.

In some embodiments, the polynucleotide further comprises a transgene or a polynucleotide encoding a transgene. In some embodiments, the polynucleotide further comprises a marker gene, a therapeutic gene, a biologic, or a nanoparticle.

Another aspect of the disclosure is directed to a method for inserting a polynucleotide, a nucleoprotein complex, a nanoparticle or a plant viral nanoparticle into the vascular system of a plant, comprising injecting the polynucleotide, nucleoprotein complex, nanoparticle or plant viral nanoparticle into a stem or a petiole of the plant, thereby inserting it into the vascular system of the cell, wherein the polynucleotide further comprises a transgene or a polynucleotide encoding a transgene, optionally wherein the polynucleotide further comprises a marker gene, a therapeutic gene, a biologics, or a nanoparticle.

Another aspect of the disclosure is directed to a method for inserting a polynucleotide, a nucleoprotein complex, a nanoparticle or a plant viral nanoparticle into the vascular system of a plant, comprising injecting the polynucleotide, nucleoprotein complex, nanoparticle or plant viral nanoparticle into a stem or a petiole of the plant, thereby inserting it into the vascular system of the cell, wherein the polynucleotide further comprises a transgene or a polynucleotide encoding a transgene, optionally wherein the polynucleotide further comprises a marker gene, a therapeutic gene, a biologics, or a nanoparticle, and wherein the needle of the syringe comprises from about 8 mm×31 G, any needle that penetrates/can penetrate the tissue can potentially be used.

Another aspect of the disclosure is directed to a method for inserting a polynucleotide, a nucleoprotein complex, a nanoparticle or a plant viral nanoparticle into the vascular system of a plant, comprising injecting the polynucleotide, nucleoprotein complex, nanoparticle or plant viral nanoparticle into a stem or a petiole of the plant, thereby inserting it into the vascular system of the cell, wherein the polynucleotide further comprises a transgene or a polynucleotide encoding a transgene, optionally wherein the polynucleotide further comprises a marker gene, a therapeutic gene, a biologics, or a nanoparticle, and wherein the needle of the syringe comprises from about 8 mm×31 G, any needle that penetrates/can penetrate the tissue can potentially be used, and wherein the syringe is loaded with from about 1 μg to about 50 μg of polynucleotide per mL of carrier.

### EXAMPLES

### Example 1: Materials and Methods

Nicotiana benthamiana Growth and Inoculation with TMV

N. benthamiana seeds were planted in Pro Mix BX soil (Greenhouse Megastore) and grown in an A1000 chamber (Conviron) with full light (˜100,000 lux), 50-60% humidity, and at 25° C. After two weeks the seedlings were transferred to larger pots, and fertilizer was administered once per week (Jack's Fertilizer #77840, JR Peters Inc.). When the plants were 4-5 weeks old, the inoculations were performed as detailed below. After visual inspection and confirmation of symptoms, leaves were harvested 10-15 days post inoculation and stored at −80° C. until further processing. Inoculations were carried out via mechanical inoculation of carborundum dusted leaves, Silwet-77 assisted spray (0.02-0.04%), and direct injection into the petiole. 3, 15 and 30 μg of purified TMV in 10 mM sodium phosphate (NaPB) buffer pH 7.4 was used, with 10 plants per treatment. Photographic documentation was carried out on days 0, 8, 10, and 15 post inoculation of TMV. In the studies native TMV was used as well as a Lys-added mutant of TMV, denotated as TMV-Lys.

### Mechanical Inoculation of TMV

Carborundum (C192-500, Thermo Fisher Scientific) was gently dusted on three leaves, and subsequently rubbed by hand with 100 μL of TMV (0.01 mg/mL, 0.5 mg/mL, 0.1 mg/mL) and NaPB buffer pH 7.4 to deliver 3 μg, 15 μg or 30 μg of TMV. Gloves were changed between each plant to avoid carryover of infectious material. Plants were kept in the dark for one hour post treatment (to avoid burning of carborundum under the grow lights), then were rinsed with tap water and placed into the plant growth chamber.

### Spray Inoculation of TMV

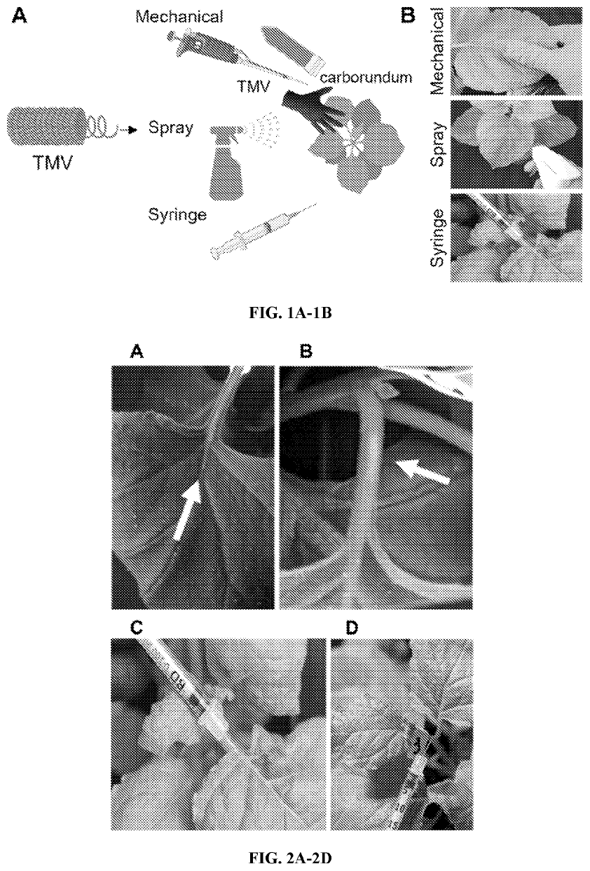

A trigger/spray nozzle (3345, Control Company) as shown in FIG. 1B was used to infect the plants. Three spraying applications were done with each one being ˜880 μL of different concentrations of TMV in NaPB buffer pH 7.4 with Silwet L-77 at varying concentrations of 0.04, 0.03, and 0.02% by volume. TMV at 0.0034 mg/mL, 0.017 mg/mL, and 0.0341 mg/mL was used to deliver 3 μg, 15 μg or 30 μg of TMV. After injection plants were immediately placed into the growth chamber—there was no need to rinse.

### Syringe Inoculation of TMV

Insulin syringes with a needle of 8 mm×31 G (328438, BD Medical Device Company) were used, and loaded with 2 μL of 1.5, 7.5, or 15 mg/mL TMV in NaPB buffer pH 7.4 to deliver 3 μg, 15 μg or 30 μg of TMV. The loading site for the syringe was tested in the stem, and petiole. After injection plants were immediately placed into the growth chamber—there was no need to rinse.

### Extraction and Purification of TMV

The extraction and purification of TMV and TMV-Lys was done as described by Bruckman and Steinmetz (2014). In brief, the leaves were homogenized in NaPB buffer using a commercial blender (6812-001, Oster), this mixture was then filtered through cheesecloth (NC9442780, Fisher Scientific), to then be centrifuged (Beckman Coulter Avanti® J-E centrifuge, 11,000 g for 20 min at 4° C.). The supernatant was then filtered through Kimwipes (21905-011, VWR), and mixed with equal parts chloroform/butanol (AC423550040/A399-4, Fisher Scientific) and mixed for 30 min at 4° C. Then this mixture was centrifuged (Beckman Coulter Avanti® J-E centrifuge, 4,500 g for 10 min at 4° C.), and the top aqueous layer containing the plant virus was taken for the next steps. The viral particles were then precipitated using 8% (w/v) PEG (MW 8000 Da) and 0.2 M NaCl (BP233-1 and BP358-212, Fisher Scientific); this mixture was then placed in a shaker overnight at 4° C. The solution was then centrifuged (Beckman Coulter Avanti® J-E centrifuge, 22,000 g for 20 min at 4° C.), and the pellet was then resuspended in 0.1 M NaPB buffer pH 7.4. Followed by another short centrifugation (Beckman Coulter Avanti® J-E centrifuge, 9,000 g for 15 min at 4° C.), the supernatant was then ultracentrifuged (Beckman Coulter Optima™ L-90 K centrifuge, 160,000 g for 3 h at 4° C.) over a 40% sucrose cushion (S0389, Sigma-Aldrich). The pellet was then left on a shaker overnight to resuspend in 10 mM NaPB buffer pH 7.4. For the final step, the solution is passed through a silica column (17-0851-01, Cytiva).

### TMV Characterization

To validate the purity and structural integrity of purified TMV, UV-vis spectroscopy, SDS-PAGE, and transmission electron microscopy (TEM) was performed.

### UV-Vis Spectroscopy

TMV concentration was determined by UV-vis spectroscopy using a NanoDrop Spectrophotometer (Thermo Fisher Scientific) and Beer-Lambert law with the extinction coefficient of TMV at 260 nm of 3 mL mg-1 cm-1.

### SDS-PAGE

For the sample preparation, 2 μg of TMV from the original purified solution were diluted to a final volume of 15 μL with NaPB buffer pH 7.4, to which 4 μL of the 4× lithium dodecyl sulfate (LDS) loading dye (Life Technologies) were added. This solution was then denatured for 5 min at 95° C., and analyzed on NOVEX NuPAGE 4-12% Bis-Tris gels (Invitrogen) in 1× morpholinepropanesulfonic acid (MOPS) buffer (ThermoFisher Scientific). SeeBlue Plus2 was as molecular standard. The gel was run at 200 V/120 mA for 40 min. Gels were stained with Commassie Brilliant Blue R-250 and imaged using an Alphalmager system (Protein Simple).

### TEM

TMV at a concentration of 0.01, 0.05, or 0.1 mg mL-1 was placed on Formvar carbon film coated TEM supports (VWR International) and stained with 2% (w/v) uranyl acetate (Agar Scientific). Images were then taken with a FEI TecnaiSpirit G2 BioTWIN TEM at 300 kV.

### Example 2: Mechanical, Spray, and Syringe Inoculation Protocols were Optimized

For mechanical inoculation established protocols were followed. For foliar spray the Silwet L-77 concentration was optimized. Silwet L-77 has been used for vacuum-assisted agroinfiltration at a concentration of 0.1% and 0.03%; for agrospray applications Silwet L-77 was used at 0.1%. Data also indicates that 0.1% Silwet L-77 was sufficient for nanoparticle delivery to tomato plants, while 0.2% and 0.3% were required for nanoparticle delivery to cotton and maize. Based on these data points, 0.02-4% Silwet L-77 spray was applied to 4-5 weeks old and 6-7 weeks old N. benthamiana plants. Using 4-5 weeks old plants and sham inoculations, it was noted that 24 h post surfactant exposure, concentrations higher than 0.04% indicated tissue damage which was evident by leaf discoloration or darkening of the leaves; higher surfactant concentrations (>0.4%) resulted in necrotic tissue. It was noted that plant age plays a role with older plants (6-7 weeks) being more robust and less necrosis observed at higher surfactant concentrations (0.4% Silwet L-77, Figure S1). For TMV infection, inoculation using 0.02% and 0.03% Silwet-L77 was tested, however only foliar spray inoculation of TMV in presence of 0.03% Silwet-L77 yielded visible symptoms (data not shown), therefore this surfactant concentration was used in all other experiments.

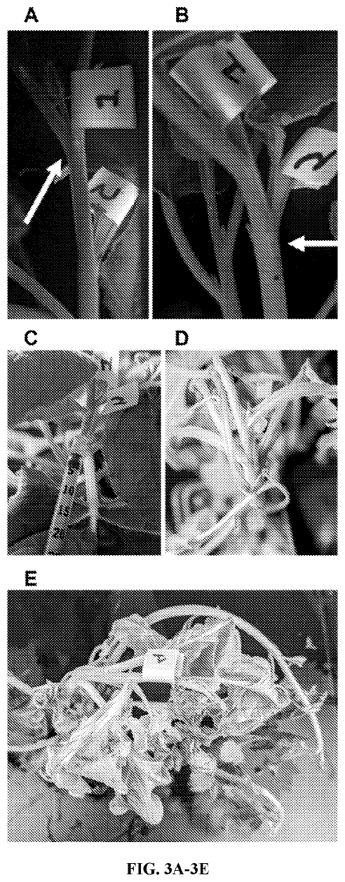

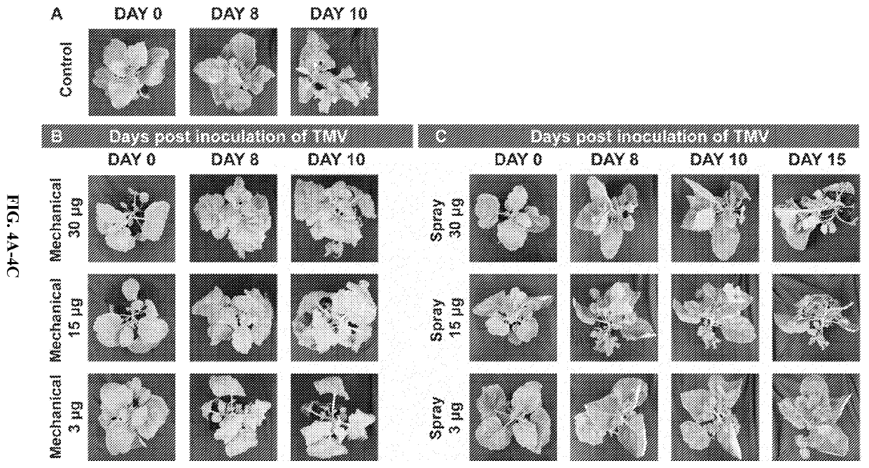

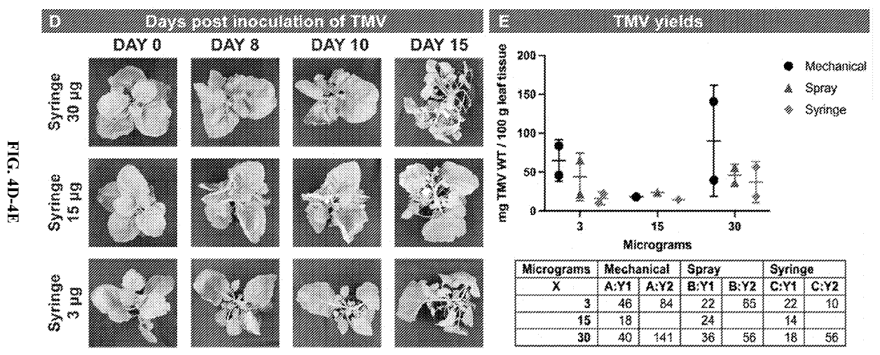

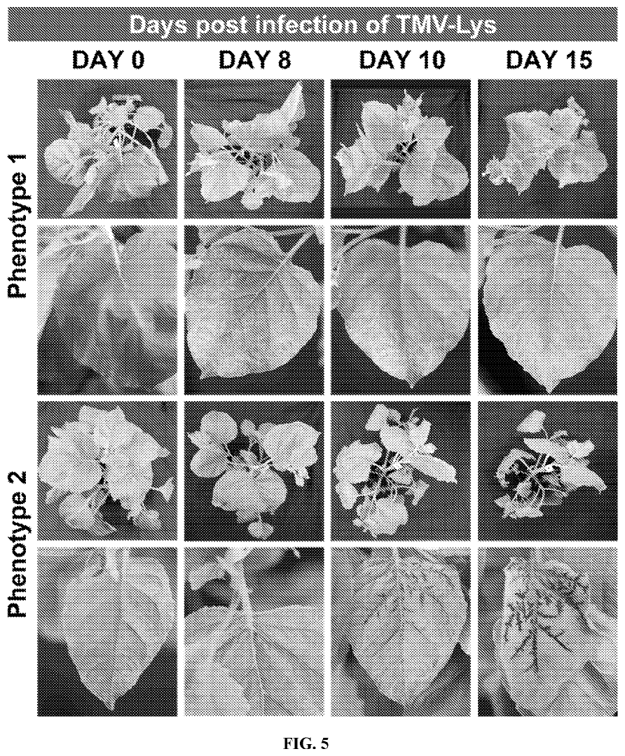

For the vascular syringe method, feasibility to administer the viral vector into the stem and petiole was tested. As a first testbed, the fluorophore Oregon Green 488™ was delivered to track the injected solution in the plant through imaging under UV light (FIG. 2A, FIG. 2B and FIG. 3A, FIG. 3B). TMV administration into the petiole resulted in systemic infection (see FIG. 4 and FIG. 5). While, dye or TMV administration into the petiole did not cause adverse effects (FIG. 2C, FIG. 2D), injection into the stem resulted in necrotic tissue observed ˜8 days post inoculation (dpi) and resulting in systemic necrosis and plant death (FIGS. 3C-3D). With this knowledge, the petiole was selected as the optimized injection site.

### Example 3: Distinct Phenotypes of TMV Infection Post Mechanical, Spray and Syringe Inoculation Method

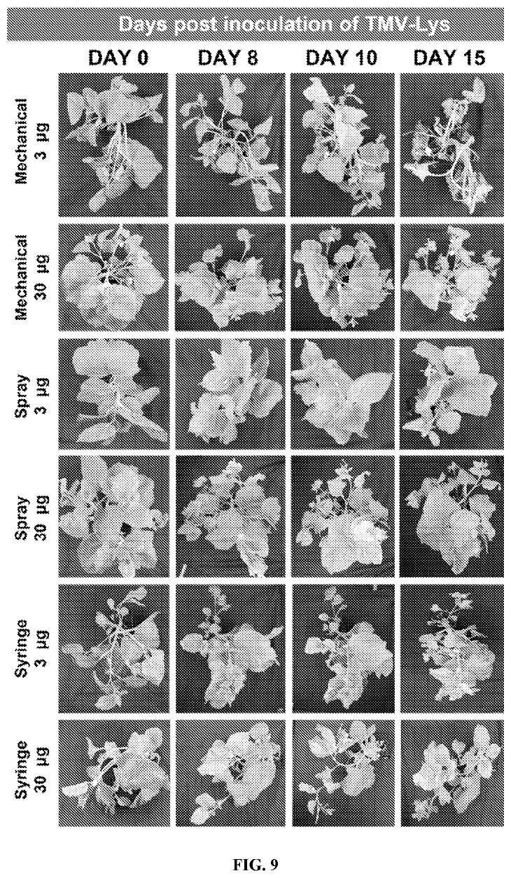

To demonstrate robustness of the methods, plants were first inoculated with TMV and then repeated the experiments using a lysine-added mutant of TMV, TMV-Lys. Photographic documentation of the TMV-infected plants is shown in FIG. 4 and TMV-Lys plants were shown in FIG. 5 and FIG. 9. The main phenotype for TMV infection includes the mosaic/mottling pattern, necrosis, uneven coloring, yellowing, and curling of leaves; however, other symptoms such as blackening of the veins have also been reported. In the studies, distinct symptoms were observed as a function of the inoculation method.

For mechanical inoculation, local symptoms appeared at 8 days post inoculation (dpi) and systemic infections were established 10+ dpi. Representative photographs are shown in FIG. 4B. The most prevalent symptom observed upon mechanical inoculation were the mosaic/mottling patterns, yellowing of leaves, and the presence of necrotic tissue. The severity of the symptoms was higher when plants were inoculated using 30 μg vs. 3 μg of TMV.

In contrast to mechanical inoculation, spray inoculated plants showed symptoms at day 10 (2 days later), and at 15+ dpi systemic infection was reported. It was observed that while traditional vacuum infiltration takes an average of 4-7 days for gene expression, gene expression is delayed when delivered via agrospray, and gene expression was confirmed 10-14 dpi. The prevalent symptoms for spray inoculation were yellowing and curling of leaves (FIG. 4C). Using higher TMV concentrations (15 and 30 μg), it was also noted some plants with blackening of veins and necrotic tissue (not shown).

Syringe inoculation showed TMV symptoms at day 10, and systemic infection at day 15. Diverse TMV symptoms were observed with two main two phenotypes: mosaic/mottling symptoms and blackening of the veins (FIG. 4D)—the latter symptoms were also observed for TMV-Lys when plants were inoculated by syringe injection into the stem (FIG. 5). For the treatments inoculated with 15 and 30 μg of TMV, the symptoms in the plants were clear, however, for 3 μg of TMV the symptoms passed almost unnoticed. The most significant difference between the various inoculation methods was the degree of variation as to whether or not systemic infection was established. While most plants showed systemic infection by 15 dpi, in some plants TMV infection was not established. Data report that TMV requires phloem loading to establish systemic infection-therefore it is likely that in plants that lacked TMV infection the injection missed the phloem. Therefore, there is room to further innovate this injection method by use of precision needles to target the phloem directly.

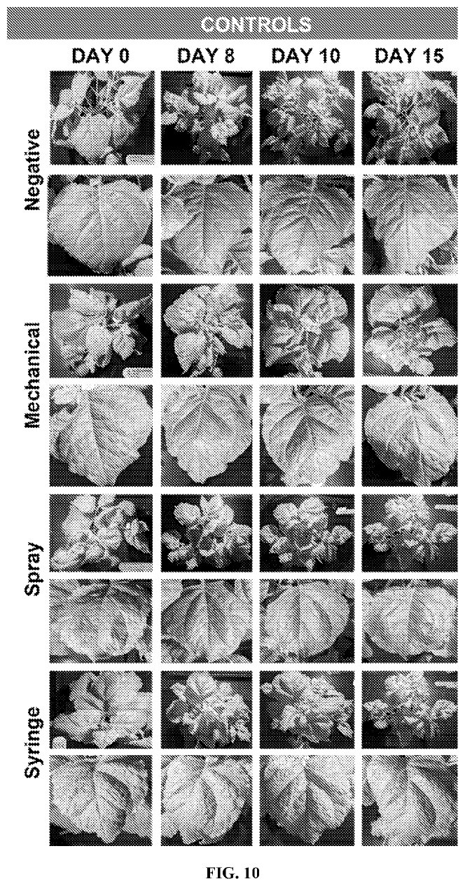

Several controls were considered: control plants were cultivated side-by-side in the same growth facility, but in a separate incubator; these plants showed no TMV infection symptoms and are shown in FIG. 4A. In addition, for each treatment a control was performed as follows: for mechanical inoculation plants were rubbed with carborundum without TMV, for spray inoculation the 0.03% Silwet L-77 was applied in NaPB buffer pH 7.4, and for the syringe inoculation NaPB buffer pH 7.4 was injected. Infection or symptoms were not apparent, and this data is shown in FIG. 10.

### Example 4: The Yields for Mechanical, Spray and Syringe Inoculation are not Statistically Different

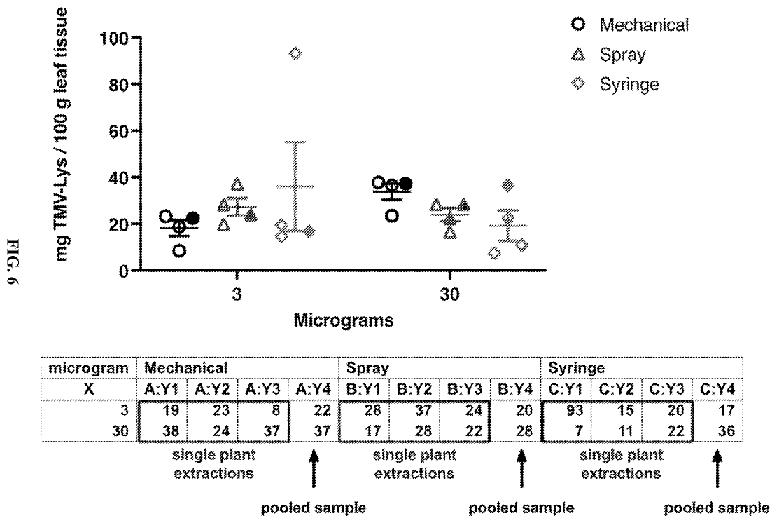

For TMV harvest, all visibly infected leaves were collected. While there was some variation between the yields comparing the three inoculation methods, there was no statistical differences between the methods (FIG. 4E—TMV; FIG. 6—TMV-Lys).

Regarding the TMV data, here leaves were pooled and 100 gram of leaf material was purified: data indicate comparable yields with a trend of increased yield when mechanical inoculation is performed at higher dose: mechanical inoculation using 3 μg vs 30 μg TMV yielded 46 vs 90 mg TMV per 100 g of infected leaf tissue—a similar trend was also apparent for the syringe method (16 vs 37 mg). In contrast, there was no dose dependence for spray inoculation using TMV yielding 43-46 mg TMV per 100 g of infected leaf tissue (however these experiments were done using a small sample size). These trends are also in agreement with the symptoms observed: overall, mechanical inoculation resulted in more severe symptoms compared to spray inoculation. Syringe-inoculated plants had a high variation of TMV symptoms, with some plants showing a severe infection while others had only mild symptoms. No noticeable differences were observed in the number of leaves that were infected per plant.

A similar trend was observed when TMV-Lys was used: the yields of TMV-Lys were compared per 100 grams of infected leaves using pooled samples, as was done for TMV, but also single plant extractions were performed and then normalized the yields to 100 g leaf tissue. The data comparing single plant extractions vs. pooled leaves are in good agreement (FIG. 6). TMV-Lys yields were comparable at either dose yielding 24-28 mg TMV-Lys per 100 g infected leaf tissue. Also, the syringe inoculation resulted in slightly lower but consistent yields of 17-19 mg TMV-Lys per 100 g infected leaf tissue. Only the mechanical inoculation showed a trend of dose-dependency doubling yields at the higher dose (34 vs. 18 mg TMV-Lys per 100 g infected leaf tissue for mechanical inoculation using 30 μg vs 3 μg TMV-Lys, FIG. 6).

Data suggests that there is no dose-dependence when the viral vectors are inoculated via foliar spray. A possible explanation is the mechanism of the surfactant. Silwet L-77 application opens up entry via the stomata and the cutical pathway, with the stomata being the main entrance. Hence, infection of TMV in N. benthamiana is dependent on the structure of the leaves, i.e., number of stomata, as well as the capacity of the surfactant to open the entry paths. Therefore, it could be speculated that regardless of the amount of TMV available in the surface of the leaf, only a certain amount would enter the intracellular environment of the plant. This is consistent with the low transfection rates reported for plasmid delivery via agrospray resulting in expression rates of 0.9-3.5%-in stark contrast, high expression rates have been reported for viral vectors reaching up to 93% efficiency; the latter can be explained by the cell-to-cell and systemic movement of the viral vector. Also, the syringe inoculation yields did not appear to be dose-dependent but success of infection was more variable.

### Example 5: The Identity of TMV is Consistent for Mechanical, Spray and Syringe Method

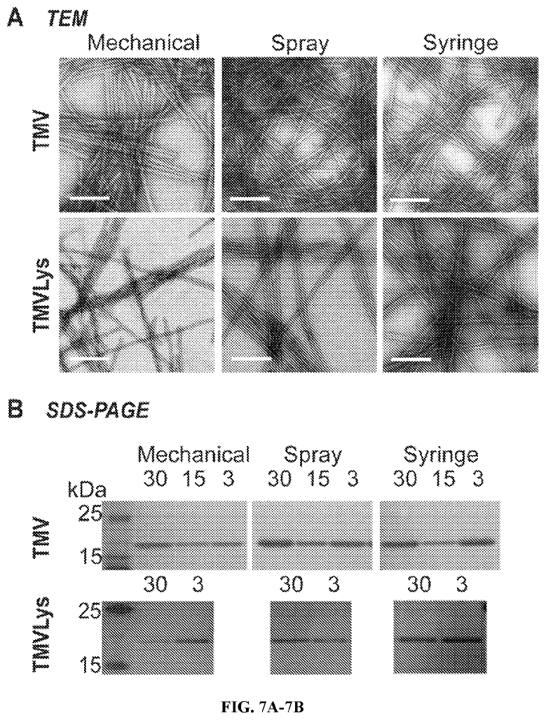

After TMV was extracted from the plants, UV-vis absorbance, SDS-PAGE and transmission electron microscopy (TEM) imaging was performed to validate the identity of TMV. UV-vis absorbance data indicate an absorbance ratio at 260/280 of 1.2, which is consistent with intact and pure TMV preparations [www.dpvweb.net] and values ranged from 1.18±0.03. TEM images show intact TMV with the typical morphology and high aspect ratio (FIG. 7A). SDS-PAGE analysis was consistent with the presence of pure TMV preparations showing the 17.5 kDa TMV coat protein; plant contaminants were not apparent; also, contamination of control plants was also not detected (FIG. 7B).

In this disclosure, mechanical vs. foliar spray vs. petiole and stem injection of TMV (and TMV-Lys) were compared as model systems for gene delivery. Successful gene delivery was measured by establishment of infection. While injection into the stem of the plant resulted in systemic toxicity and plant loss, targeting the petiole was found productive with good infection rate and yields comparable to any other method. There was variation between yields from preparation to preparation with mechanical, spray and syringe inoculation yielding 40-141 mg, 36-56 mg, 18-56 mg TMV and per 100 grams of leaves. Similar yields were obtained using TMV-Lys, with 24-38 mg, 17-28, 7-36 mg TMV-Lys and per 100 grams of leaves for mechanical, spray and syringe inoculation, respectively.

Each of these methods offer advantages-mechanical inoculation shows a high degree of reproducibility given the ease of the method. Foliar spray application is scalable and may offer a broad platform for agricultural engineering and could facilitate transient gene delivery in large extensions of crops. The syringe inoculation provides an aseptic method that may be suitable for the pharmaceutical industry. Compared with the current standard of Agrobacterium-based transformation methods, syringe inoculation does not require the culturing of gram-negative bacteria that i) can be affected by epigenetic variation, ii) introduce LPS into the product which then requires additional purification steps, and iii) adds complexity to the manufacturing set up, requiring plants and a fermenter. While agrobacteria-based transformation is the effective and currently industry-standard, approaches such as syringe inoculation provides viable and more effective alternatives.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Barta, A., Sommergruber, K., Thompson, D., Hartmuth, K., Matzke, M. A., Matzke, A. J. M., 1986. The expression of a nopaline synthase-human growth hormone chimaeric gene in transformed tobacco and sunflower callus tissue, Plant Molecular Biology.
2. Bruckman, M. A., Steinmetz, N. F., 2014. Chemical Modification of the Inner and Outer Surfaces of Tobacco Mosaic Virus (TMV), in: Virus Hybrids as Nanomaterials. Methods in Molecular Biology. Humana Press, Totowa, pp. 173-185. https://doi.org/10.1007/978-1-62703-751-8_13
3. Cai, H., Shukla, S., Wang, C., Masarapu, H., Steinmetz, N. F., 2019. Heterologous Prime-Boost Enhances the Antitumor Immune Response Elicited by Plant-Virus-Based Cancer Vaccine. J Am Chem Soc 141, 6509-6518. https://doi.org/10.1021/jacs.9b01523
4. Cao, Y., Lim, E., Xu, M., Weng, J. K., Marelli, B., 2020. Precision Delivery of Multiscale Payloads to Tissue-Specific Targets in Plants. Advanced Science 7. https://doi.org/10.1002/advs.201903551
5. Cheng, N. H., Su, C. L., Carter, S. A., Nelson, R. S., 2000. Vascular invasion routes and systemic accumulation patterns of tobacco mosaic virus in Nicotiana benthamiana. Plant Journal 23, 349-362. https://doi.org/10.1046/j.1365-313X.2000.00788.x
6. Chung, Y. H., Cai, H., Steinmetz, N. F., 2020. Viral nanoparticles for drug delivery, imaging, immunotherapy, and theranostic applications. Advanced Drug Delivery Reviews. https://doi.org/10.1016/j.addr.2020.06.024
7. Chung, Y. H., Church, D., Koellhoffer, E. C., Osota, E., Shukla, S., Rybicki, E. P., Pokorski, J. K., Steinmetz, N. F., 2021. Integrating plant molecular farming and materials research for next-generation vaccines. Nature Reviews Materials. https://doi.org/10.1038/s41578-021-00399-5
8. Demirer, G. S., Zhang, H., Matos, J. L., Goh, N. S., Cunningham, F. J., Sung, Y., Chang, R., Aditham, A. J., Chio, L., Cho, M. J., Staskawicz, B., Landry, M. P., 2019. High aspect ratio nanomaterials enable delivery of functional genetic material without DNA integration in mature plants. Nature Nanotechnology 14, 456-464. https://doi.org/10.1038/s41565-019-0382-5
9. Fister, A. S., Landherr, L., Maximova, S. N., Guiltinan, M. J., 2018. Transient expression of CRISPR/Cas9 machinery targeting TcNPR3 enhances defense response in Theobroma cacao. Frontiers in Plant Science 9. https://doi.org/10.3389/fpls.2018.00268
10. Gad, M. A., Li, M., Ahmed, F. K., Almoammar, H., 2020. Nanomaterials for gene delivery and editing in plants: Challenges and future perspective. Multifunctional Hybrid Nanomaterials for Sustainable Agri-Food and Ecosystems 135-153. https://doi.org/10.1016/b978-O-12-821354-4.00006-6
11. Geiger, F. C., Eber, F. J., Eiben, S., Mueller, A., Jeske, H., Spatz, J. P., Wege, C., 2013. TMV nanorods with programmed longitudinal domains of differently addressable coat proteins. Nanoscale 5, 3808-3816. https://doi.org/10.1039/c3nr33724c
12. Hahn, S., Giritch, A., Bartels, D., Bortesi, L., Gleba, Y., 2015. A novel and fully scalable Agrobacterium spray-based process for manufacturing cellulases and other cost-sensitive proteins in plants. Plant Biotechnology Journal 13, 708-716. https://doi.org/10.1111/pbi.12299
13. Hiatt, A., Caffferkey, R., Bowdish, K., 1989. Production of antibodies in transgenic plants. Nature 342, 76-78.
14. Hu, P., An, J., Faulkner, M. M., Wu, H., Li, Z., Tian, X., Giraldo, J. P., 2020a. Nanoparticle Charge and Size Control Foliar Delivery Efficiency to Plant Cells and Organelles. ACS Nano 14, 7970-7986. https://doi.org/10.1021/acsnano.9b09178
15. Hu, P., An, J., Faulkner, M. M., Wu, H., Li, Z., Tian, X., Giraldo, J. P., 2020b. Nanoparticle Charge and Size Control Foliar Delivery Efficiency to Plant Cells and Organelles. ACS Nano 14, 7970-7986. https://doi.org/10.1021/acsnano.9b09178
16. Hull, R., 2009. Mechanical inoculation of plant viruses. Current Protocols in Microbiology. https://doi.org/10.1002/9780471729259.mc16b06s13
17. Kagale, S., Uzuhashi, S., Wigness, M., Bender, T., Yang, W., Hossein Borhan, M., Rozwadowski, K., 2012. TMV-Gate vectors: Gateway compatible tobacco mosaic virus based expression vectors for functional analysis of proteins. Scientific Reports 2. https://doi.org/10.1038/srep00874
18. Kaur, M., Manchanda, P., Kalia, A., Ahmed, F. K., Nepovimova, E., Kuca, K., Abd-Elsalam, K. A., 2021. Agroinfiltration mediated scalable transient gene expression in genome edited crop plants. International Journal of Molecular Sciences. https://doi.org/10.3390/ijms221910882
19. Kwak, S. Y., Lew, T. T. S., Sweeney, C. J., Koman, V. B., Wong, M. H., Bohmert-Tatarev, K., Snell, K. D., Seo, J. S., Chua, N. H., Strano, M. S., 2019. Chloroplast-selective gene delivery and expression in planta using chitosan-complexed single-walled carbon nanotube carriers. Nature Nanotechnology 14, 447-455. https://doi.org/10.1038/s41565-019-0375-4
20. Landry, M. P., Mitter, N., 2019. How nanocarriers delivering cargos in plants can change the GMO landscape. Nature Nanotechnology 14, 512-514. https://doi.org/10.1038/s41565-019-0463-5
21. Liu, Q., Chen, B., Wang, Q., Shi, X., Xiao, Z., Lin, J., Fang, X., 2009. Carbon nanotubes as molecular transporters for walled plant cells. Nano Letters 9, 1007-1010. https://doi.org/10.1021/n1803083u
22. Marillonnet, S., Giritch, A., Gils, M., Kandzia, R., Klimyuk, V., Gleba, Y., 2004. In planta engineering of viral RNA replicons: Efficient assembly by recombination of DNA modules delivered by Agrobacterium.
23. McNulty, M. J., Xiong, Y., Yates, K., Karuppanan, K., Hilzinger, J. M., Berliner, A. J., Delzio, J., Arkin, A. P., Lane, N. E., Nandi, S., McDonald, K. A., 2021. Molecular pharming to support human life on the moon, mars, and beyond. Critical Reviews in Biotechnology. https://doi.org/10.1080/07388551.2021.1888070
24. O'Callaghan, M., 2016. Microbial inoculation of seed for improved crop performance: issues and opportunities. Applied Microbiology and Biotechnology. https://doi.org/10.1007/s00253-016-7590-9
25. Ortega-Rivera, O. A., Shin, M. D., Chen, A., Beiss, V., Moreno-Gonzalez, M. A., Lopez-Ramirez, M. A., Reynoso, M., Wang, H., Hurst, B. L., Wang, J., Pokorski, J. K., Steinmetz, N. F., 2021. Trivalent Subunit Vaccine Candidates for COVID-19 and Their Delivery Devices. J Am Chem Soc 143, 14748-14765. https://doi.org/10.1021/jacs. 1c06600
26. Palmer, K., Gleba, Y., 2013. Current Topics in Microbiology and Immunology Plant Viral Vectors. Springer, Berlin. https://doi.org/https://doi.org/10.1007/82_2013_352
27. Royal, J. M., Simpson, C. A., McCormick, A. A., Phillips, A., Hume, S., Morton, J., Shepherd, J., Oh, Y., Swope, K., Debeauchamp, J. L., Webby, R. J., Cross, R. W., Borisevich, V., Geisbert, T. W., Demarco, J. K., Bratcher, B., Haydon, H., Pogue, G. P., 2021. Development of a sars-cov-2 vaccine candidate using plant-based manufacturing and a tobacco mosaic virus-like nano-particle. Vaccines (Basel) 9. https://doi.org/10.3390/vaccines9111347
28. Scholthof, K. B. G., 2004. Tobacco Mosaic Virus: A model system for plant biology. Annual Review of Phytopathology. https://doi.org/10.1146/annurev.phyto.42.040803.140322
29. Shin, M. D., Shukla, S., Chung, Y. H., Beiss, V., Chan, S. K., Ortega-Rivera, O. A., Wirth, D. M., Chen, A., Sack, M., Pokorski, J. K., Steinmetz, N. F., 2020. COVID-19 vaccine development and a potential nanomaterial path forward. Nature Nanotechnology. https://doi.org/10.1038/s41565-020-0737-y
30. Torti, S., Schlesier, R., Thümmler, A., Bartels, D., Römer, P., Koch, B., Werner, S., Panwar, V., Kanyuka, K., Wiren, N. von, Jones, J. D. G., Hause, G., Giritch, A., Gleba, Y., 2021. Transient reprogramming of crop plants for agronomic performance. Nature Plants 7, 159-171. https://doi.org/10.1038/s41477-021-00851-y
31. van Dijk, M., Morley, T., Rau, M. L., Saghai, Y., 2021. A meta-analysis of projected global food demand and population at risk of hunger for the period 2010-2050. Nature Food 2, 494-501. https://doi.org/10.1038/s43016-021-00322-9
32. Vojta, L., Ljuma-Skupnjak, L., Budimir, A., Vukičević, S., Fulgosi, H., 2015. Rapid transient expression of human granulocyte-macrophage colony-stimulating factor in two industrial cultivars of tobacco (Nicotiana tabacum L.) by agroinfiltration. Biotechnology Reports 7, 81-86. https://doi.org/10.1016/j.btre.2015.05.006
33. Wang, B., Huang, J., Zhang, M., Wang, Y., Wang, H., Ma, Y., Zhao, X., Wang, X., Liu, C., Huang, H., Liu, Y., Lu, F., Yu, H., Shao, M., Kang, Z., 2020. Carbon Dots Enable Efficient Delivery of Functional DNA in Plants. ACS Applied Bio Materials 3, 8857-8864. https://doi.org/10.1021/acsabm.0c01170
34. Wang, C., Beiss, V., Steinmetz, N. F., 2019. Cowpea Mosaic Virus Nanoparticles and Empty Virus-Like Particles Show Distinct but Overlapping Immunostimulatory Properties. J Virol 93. https://doi.org/10.1128/JVI
35. Wang, Y. di, Li, F., Zhang, X., Liu, T. li, Liang, W. xing, Li, D. long, 2022. PnSCR82, a small cysteine-rich secretory protein of Phytophthora nicotianae, can enhance defense responses in plants. Journal of Integrative Agriculture 21, 751-761. https://doi.org/10.1016/S2095-3119 (21) 63681-X
36. Ward, B. J., Gobeil, P., Seguin, A., Atkins, J., Boulay, I., Charbonneau, P. Y., Couture, M., D'Aoust, M. A., Dhaliwall, J., Finkle, C., Hager, K., Mahmood, A., Makarkov, A., Cheng, M. P., Pillet, S., Schimke, P., St-Martin, S., Trepanier, S., Landry, N., 2021. Phase 1 randomized trial of a plant-derived virus-like particle vaccine for COVID-19. Nature Medicine 27, 1071-1078. https://doi.org/10.1038/s41591-021-01370-1
37. Ward, B. J., Makarkov, A., Séguin, A., Pillet, S., Trépanier, S., Dhaliwall, J., Libman, M. D., Vesikari, T., Landry, N., 2020. Efficacy, immunogenicity, and safety of a plant-derived, quadrivalent, virus-like particle influenza vaccine in adults (18-64 years) and older adults (>65 years): two multicentre, randomised phase 3 trials. The Lancet 396, 1491-1503. https://doi.org/10.1016/S0140-6736 (20) 32014-6
38. Zhang, Youjun, Chen, M., Siemiatkowska, B., Toleco, M. R., Jing, Y., Strotmann, V., Zhang, J., Stahl, Y., Fernie, A. R., 2020. A Highly Efficient Agrobacterium-Mediated Method for Transient Gene Expression and Functional Studies in Multiple Plant Species. Plant Communications 1. https://doi.org/10.1016/j.xplc.2020.100028
39. Zhang, Yilin, Yan, J., Avellan, A., Gao, X., Matyjaszewski, K., Tilton, R. D., Lowry, G. v., 2020. Temperature- And pH-Responsive Star Polymers as Nanocarriers with Potential for in Vivo Agrochemical Delivery. ACS Nano 14, 10954-10965. https://doi.org/10.1021/acsnano.0c03140
40. Zhang, Y. F., Li, D. P., Jin, X., Huang, Z., 2014. Fighting Ebola with ZMapp: spotlight on plant-made antibody. Science China Life Sciences 57, 987-988. https://doi.org/10.1007/s11427-014-4746-7
41. Zhu, H., Li, C., Gao, C., 2020. Applications of CRISPR-Cas in agriculture and plant biotechnology. Nature Reviews Molecular Cell Biology 21, 661-677. doi.org/10.1038/s41580-020-00288-9

1. A method for inserting a polynucleotide, a nucleoprotein complex, a nanoparticle or a plant viral nanoparticle into a vascular system of a plant, comprising injecting the polynucleotide, nucleoprotein complex, nanoparticle or plant viral nanoparticle into a stem or a petiole of the plant, thereby inserting it into the vascular system of the plant.

2. The method of claim 1, wherein the polynucleotide is DNA or RNA.

3. The method of claim 1, wherein the polynucleotide comprises a nucleoprotein complex, a nanoparticle or plant viral nanoparticle, optionally a plasmid/protein complex, plasmid, polymer-condensed plasmid, or viral vector, with an optional linker, further optionally a Lys linker.

4. The method of claim 3, wherein the viral vector is or is derived from tobacco mosaic virus (TMV), cowpea mosaic virus, or potato virus X.

5. The method of claim 3, wherein the plasmid is or is derived from an agrobacterium Ti (tumor inducing) plasmid, a plant viral-derived expression plasmid (based on tobacco mosaic virus (TMV), cowpea mosaic virus or potato virus X).

6. The method of claim 1, wherein the polynucleotide further comprises a transgene or a polynucleotide encoding a transgene, optionally wherein the polynucleotide further comprises a marker gene, a therapeutic gene, a biologics, or a nanoparticle.

7. The method of claim 1, wherein the polynucleotide further comprises a transgene or a polynucleotide encoding a transgene, optionally wherein the polynucleotide further comprises a marker gene, a therapeutic gene, a biologics, or a nanoparticle, and wherein the needle of the syringe comprises from about 8 mm×31 G, any needle that penetrates/can penetrate the tissue can potentially be used.

8. The method of claim 1, wherein the polynucleotide further comprises a transgene or a polynucleotide encoding a transgene, optionally wherein the polynucleotide further comprises a marker gene, a therapeutic gene, a biologics, or a nanoparticle, and wherein the needle of the syringe comprises from about 8 mm×31 G, any needle that penetrates/can penetrate the tissue can potentially be used, and wherein the syringe is loaded with from about 1 μg to about 50 μg of polynucleotide per mL of carrier.

9. The method of claim 1, wherein the polynucleotide further comprises a transgene or a polynucleotide encoding a transgene, optionally wherein the polynucleotide is selected from a marker gene, a therapeutic gene, or a biologic.

10. The method of claim 1, wherein the plant is N. benthamiana, N. tabacum, or black eyed peas, or Arabidopsis thaliana.

11. The method of claim 1, wherein the injection of the polynucleotide comprises contacting and inserting the polynucleotide, nucleoprotein complex, or plant viral nanoparticle with a needle syringe into the vascular of the plant.

12. The method of claim 11, wherein the needle of the syringe comprises from about 8 mm×31 G, any needle that penetrates/can penetrate the tissue can potentially be used.

13. The method of claim 11, wherein the syringe is loaded with from about 1 μg to about 50 μg of polynucleotide per mL of carrier.

14. The method of claim 13, wherein the carrier comprises a buffer solution at a pH of from about 7.2 to about 7.6, or alternatively about 7.4.

15. The method of claim 1, further comprising growing the plant until expression is established, 3-10 days post inoculation with polynucleotide or nucleoprotein complex or 7-20 days when inoculating with a nanoparticle or plant viral nanoparticle.

16. The method of claim 15, further comprising isolating the polynucleotide, nucleoprotein complex, or plant viral nanoparticle from the plant.


SEQUENCE LISTING (5 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 3 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 4 - protein, 188 aa, Physalis mottle virus
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 5 - protein, 268 aa, Sesbania mosaic virus
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN